(19) United States
(12) Patent Application Publication
(10) Pub. No.: US 2015/0087613 A1
(43) Pub. Date: Mar. 26, 2015

(54) Inhibition of Opioid Antinociceptive Tolerance and Withdrawal in Nociceptive Pain Therapy

(71) Applicant: Saint Louis University, St. Louis, MO (US)

(72) Inventor: Daniela SALVEMINI, Chesterfield, MO (US)

(21) Appl. No.: 14/496,030

(22) Filed: Sep. 25, 2014

Related U.S. Application Data
(60) Provisional application No. 61/882,812, filed on Sep. 26, 2013.

(51) Int. Cl. A61K 31/7076; A61K 31/485

(52) U.S. Cl. CPC A61K 31/7076; A61K 31/485
USPC 514/45

(57) ABSTRACT

The invention provides methods and compositions for inhibiting opioid tolerance by administering an A₃AR agonist to a subject receiving opiate therapy for nociceptive pain. Also provided are methods of treating opiate withdrawal using an A₃AR agonist.

The present application claims benefit of priority to U.S. Provisional Application Ser. No. 61/882,812, filed Sep. 26, 2013, the entire contents of which are hereby incorporated by reference.

### BACKGROUND OF THE INVENTION

1. Field of the Invention

This invention relates to the fields of medicine and cell biology. More specifically, it relates to the blocking of opioid-induced hyperalgesia and antinociceptive tolerance using A3AR agonists.

2. Related Art

A devastating health problem in the United States is the inadequate treatment of pain. One third of all Americans suffer from some form of chronic pain, and a third of these have pain, which is resistant to current medical therapy. The economic impact of pain is equally large at approximately $100 billion annually (Renfry, 2003). Opioid/narcotic analgesics, typified by morphine, are the most effective treatments for acute and chronic severe pain. However their clinical utility is often hampered by the development of analgesic tolerance which requires escalating doses to achieve equivalent pain relief (Foley, 1995). This complex pathophysiological cycle represents a critical barrier to the quality of life of these patients due to the resulting drug-induced sedation, reduced physical activity, constipation, respiratory depression, high potential for addiction, and other side-effects (Foley, 1995). While progress has been made in modifying this drug class to improve their formulated delivery, pharmacokinetics, and potential for abuse, little progress has been made in preventing the development of antinociceptive tolerance. Accordingly, there is major interest in new approaches to maintain opioid efficacy during repetitive dosing for chronic pain without engendering tolerance or unacceptable side-effects.

The purine nucleoside adenosine, as well as its derivatives, generated by metabolically stressed or inflamed cells and tissues exhibits diverse and potent physiological responses on most organs and tissues, including the central nervous system. Rising adenosine concentrations signal a threat to local homeostasis and initiate a myriad of responses in nearby neurons, astrocytes and microglia cells. The actions of adenosine are mediated through G-protein-coupled receptors, which are classified into four subtypes, A₁, A_{2A}, A_{2B}, and A₃, on the basis of their affinity order profiles for agonists and antagonists (Freholm et al., 2011). These receptor subtypes are characterized by their capacity to either increase or decrease intracellular cAMP levels (Fredholm et al., 2001). A₁ and A₃ ARs, coupled through Gᵢ protein, mediate biological effects opposite to A_{2A} and A_{2B} ARs, which are coupled to Gₛ proteins (Fredholm et al., 2001).

### SUMMARY OF THE INVENTION

Thus, in accordance with the present invention, there is provided a method of reducing opioid antinociceptive tolerance in a subject receiving opiate therapy for acute/severe and chronic neuropathic (malignant and non malignant) pain comprising administering to the subject an amount of an A₃AR agonist sufficient to reduce opioid antinociceptive tolerance. The opioid may be morphine, oxycodone, or fentanyl. The A₃AR agonist may be IB-MECA or MRS5698. The subject may be a human or a non-human mammal. The nociceptive pain may be chronic pain or acute pain. The nociceptive pain may be the result of an injury, such as a penetration wound, a burn, frostbite or a fracture. The nociceptive pain may be the result of a disease, such as diabetes, postsurgical pain, bone cancer pain, spinal nerve injuries, multiple sclerosis, arthritis, an autoimmune disease, or an infection.

The opiate and A₃AR agonist may be delivered at the same time, and may be co-formulated or not co-formulated. Alternatively, the opiate and the A₃AR agonist may be delivered at distinct times, such as where the opioid is delivered before the A₃AR agonist, or after the A₃AR agonist. The opiate and A₃AR agonist may be delivered in alternating administrations. The A₃AR agonist and/or the opiate may be delivered over a period of one week, two weeks, three weeks, four weeks, one month, two months, three months, four months, five months, six months, seven months, eight months, nine months, ten months, eleven months, one year, two years or three years. The opiate and/or the A₃AR agonist may be delivered by continuous infusion, such as by an implanted pump.

In another embodiment, there is provided a method of preventing or treating opioid withdrawal in a subject receiving opiates comprising administering to the subject an amount of an A₃AR agonist sufficient to treat one or more symptoms of opioid withdrawal. The opioid may be morphine, oxycodone, fentanyl, cocaine heroin, or opium. The A₃AR agonist may be IB-MECA or MRS5698. The subject may be is a human or a non-human mammal. The patient may have received treatment for pain, such as chronic or acute pain. The acute pain may be the result of an injury, such as a penetration wound, a burn, frostbite or a fracture. The chronic pain is the result of a disease, such as arthritis, an autoimmune disease, or an infection.

The A₃AR agonist may be delivered prior to initiating withdrawal or after initiating withdrawal. The A₃AR agonist may be co-administered with a decreasing dosage of opiate. The A₃AR agonist may be delivered prior to beginning opiate therapy. The A₃AR agonist may be delivered for a period of time after the opiate is no longer administered to the subject. The A₃AR agonist may be delivered over a period of one week, two weeks, three weeks, four weeks, one month, two months, three months, four months, five months, or six months after the opiate is no longer administered to the subject. The opiate and/or the A₃AR agonist may be delivered by continuous infusion, such as by an implanted pump.

The subject may be an abuser of illegal or illegally obtained opiate. The one or more symptoms may comprise agitation, anxiety, muscle ache, increased tearing, insomnia, runny nose, sweating, and yawning, while late symptoms of withdrawal include abdominal cramping, diarrhea, dilated pupils, goose bumps, nausea and/or vomiting. The method may further comprise subjecting the subject to a drug treatment program, such as methadone treatment or buprenorphine treatment.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed.

FIG. 1. IB-MECA blocks morphine-induced antinociceptive tolerance. Intraperitoneal injection of IB-MECA blocked the development of morphine-induced antinociceptive tolerance. Data expressed as mean±standard deviation of n=2 rats. Data were analyzed by two-tailed, two-way ANOVA using Bonferroni comparisons to t₀ or Morphine-vehicle. Significance was accepted at P<0.05. Mean±SD; *P<0.05 and ***P<0.001 vs. t0; †P<0.05 and †††P<0.001 vs. time-matched Morphine+Veh, ANOVA with Bonferroni, n=2.

FIGS. 2A-C. IB-MEC attenuates naloxone precipitated withdrawal behaviours in morphine dependent mice during a 30 minute observation. (FIG. 2A) Co-administration of IB-MECA (0.1 mg/kg) with a three day dependence regimen of morphine significantly protected male Balb/c mice from the classic naloxone precipitated opioid withdrawal jumping behaviour during the standard 30 minute observation period post precipitation of withdrawal (p<0.05). (FIG. 2B) Front paw shakes and (FIG. 2C) Hunched prayer position behaviours were also significantly attenuated by coadministration of IB-MECA in the same animals. * P<0.05, *** P<0.001 as assessed using an unpaired Students t-test. Data are expressed as mean+/−Standard error of the mean. n=7-8 per group.

FIG. 3. MRS 5698 at non-analgesic doses prevents the development of morphine tolerance. When given at peak pain (D7) in CCI rats, repeated injections of morphine (10.5 μmol/kg; s.c.; □) loses their analgesic effect by D10 with complete loss by D12. When a non-analgesic dose of MRS5698 (0.18 μmol/k g; s.c.; ) is administered as a cocktail with morphine (10.5 μmol/kg; s.c.; ▪) morphine retains is analgesic effects through D13, Mean±SD of n=4 mice. Data are analyzed by two-way ANOVA with Dunnett's post hoc test. # P<0.001 D7_{BL} vs. D0; * P<0.001 tₕ vs. D7_{BL}.

### DESCRIPTION OF ILLUSTRATIVE EMBODIMENTS

The results reported here establish, for the first time, the use of A₃AR agonists to block the development of morphine-induced antinociceptive tolerance, providing a novel mechanistic rationale for development of A₃AR agonist as adjunct to opioids in pain management. In addition, the results show that the A₃AR agonist IB-MECA blocks morphine-induced withdrawal. Results from these studies will most certainly lead to novel mechanism-based therapeutic strategies based on A₃AR targeting to block morphine antinociceptive tolerance and morphine withdrawal addressing huge unmet unmet need.

### I. PAIN

Pain is an unpleasant feeling often caused by intense or damaging stimuli. The International Association for the Study of Pain's widely used definition states: “Pain is an unpleasant sensory and emotional experience associated with actual or potential tissue damage, or described in terms of such damage.”

Pain motivates the individual to withdraw from damaging situations, to protect a damaged body part while it heals, and to avoid similar experiences in the future. Most pain resolves promptly once the painful stimulus is removed and the body has healed, but sometimes pain persists despite removal of the stimulus and apparent healing of the body; and sometimes pain arises in the absence of any detectable stimulus, damage or disease.

Pain is the most common reason for physician consultation in the United States. It is a major symptom in many medical conditions, and can significantly interfere with a person's quality of life and general functioning. Psychological factors such as social support, hypnotic suggestion, excitement, or distraction can significantly modulate pain's intensity or unpleasantness.

The International Association for the Study of Pain (IASP) has classified pain according to specific characteristics: (a) region of the body involved (e.g., abdomen, lower limbs), (b) system whose dysfunction may be causing the pain (e.g., nervous, gastrointestinal), (c) duration and pattern of occurrence, (d) intensity and time since onset, and (e) etiology. This system has been criticized by Clifford J. Woolf and others as inadequate for guiding research and treatment. According to Woolf, there are three classes of pain: nociceptive pain (see hereunder), inflammatory pain which is associated with tissue damage and the infiltration of immune cells, and pathological pain which is a disease state caused by damage to the nervous system (neuropathic pain, see hereunder) or by its abnormal function (dysfunctional pain, like in fibromyalgia, irritable bowel syndrome, tension type headache, etc.).

A. Chronic Pain

Pain is usually transitory, lasting only until the noxious stimulus is removed or the underlying damage or pathology has healed, but some painful conditions, such as rheumatoid arthritis, peripheral neuropathy, cancer and idiopathic pain, may persist for years. Pain that lasts a long time is called chronic, and pain that resolves quickly is called acute. Traditionally, the distinction between acute and chronic pain has relied upon an arbitrary interval of time from onset; the two most commonly used markers being 3 months and 6 months since the onset of pain, though some theorists and researchers have placed the transition from acute to chronic pain at 12 months. Others apply acute to pain that lasts less than 30 days, chronic to pain of more than six months duration, and subacute to pain that lasts from one to six months. A popular alternative definition of chronic pain, involving no arbitrarily fixed durations is “pain that extends beyond the expected period of healing.” Chronic pain may be classified as cancer pain or benign.

B. Nociceptive Pain

Nociceptive pain is caused by stimulation of peripheral nerve fibers that respond only to stimuli approaching or exceeding harmful intensity (nociceptors), and may be classified according to the mode of noxious stimulation; the most common categories being “thermal” (heat or cold), “mechanical” (crushing, tearing, etc.) and “chemical” (iodine in a cut, chili powder in the eyes). As subset of nocicipetive pain is called “inflammatory” pain, as it results from tissue damage and the response of innate inflammatory responses. Nociceptive pain may also be divided into “visceral,” “deep somatic” and “superficial somatic” pain. Visceral structures are highly sensitive to stretch, ischemia and inflammation, but relatively insensitive to other stimuli that normally evoke pain in other structures, such as burning and cutting. Visceral pain is diffuse, difficult to locate and often referred to a distant, usually superficial, structure. It may be accompanied by nausea and vomiting and may be described as sickening, deep, squeezing, and dull. Deep somatic pain is initiated by stimulation of nociceptors in ligaments, tendons, bones, blood vessels, fasciae and muscles, and is dull, aching, poorly localized pain. Examples include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns.

C. Neuropathic Pain

Neuropathic pain is pain caused by damage or disease that affects the somatosensory system. It may be associated with abnormal sensations called dysesthesia, and pain produced by normally non-painful stimuli (allodynia). Neuropathic pain may have continuous and/or episodic (paroxysmal) components. The latter are likened to an electric shock. Common qualities include burning or coldness, “pins and needles” sensations, numbness and itching. Nociceptive pain, by contrast, is more commonly described as aching.

Neuropathic pain may result from disorders of the peripheral nervous system or the central nervous system (brain and spinal cord). Thus, neuropathic pain may be divided into peripheral neuropathic pain, central neuropathic pain, or mixed (peripheral and central) neuropathic pain. Central neuropathic pain is found in spinal cord injury, multiple sclerosis, and some strokes. Aside from diabetes (see diabetic neuropathy) and other metabolic conditions, the common causes of painful peripheral neuropathies are herpes zoster infection, HIV-related neuropathies, nutritional deficiencies, toxins, remote manifestations of malignancies, immune mediated disorders and physical trauma to a nerve trunk.

Neuropathic pain is common in cancer as a direct result of cancer on peripheral nerves (e.g., compression by a tumor), or as a side effect of chemotherapy, radiation injury or surgery. After a peripheral nerve lesion, aberrant regeneration may occur. Neurons become unusually sensitive and develop spontaneous pathological activity, abnormal excitability, and heightened sensitivity to chemical, thermal and mechanical stimuli. This phenomenon is called “peripheral sensitization.”

The (spinal cord) dorsal horn neurons give rise to the spinothalamic tract (STT), which constitutes the major ascending nociceptive pathway. As a consequence of ongoing spontaneous activity arising in the periphery, STT neurons develop increased background activity, enlarged receptive fields and increased responses to afferent impulses, including normally innocuous tactile stimuli. This phenomenon is called central sensitization. Central sensitization is an important mechanism of persistent neuropathic pain.

Other mechanisms, however, may take place at the central level after peripheral nerve damage. The loss of afferent signals induces functional changes in dorsal horn neurons. A decrease in the large fiber input decreases activity of interneurons inhibiting nociceptive neurons, i.e., loss of afferent inhibition. Hypoactivity of the descending antinociceptive systems or loss of descending inhibition may be another factor. With loss of neuronal input (deafferentation) the STT neurons begin to fire spontaneously, a phenomenon designated “deafferentation hypersensitivity.” Neuroglia (“glial cells”) may play a role in central sensitization. Peripheral nerve injury induces glia to release proinflammatory cytokines and glutamate—which, in turn influence neurons.

D. Current Therapies

The following is a discussion of different therapies currently applied against nociceptive pain conditions. Such is exemplary and not limiting. Currently, there are a wide number of agents effective at treating nociceptive pain. These include salicylates, such as Aspirin (acetylsalicylic acid), Diflunisal and Salsalate, Propionic acid derivatives (Ibuprofen, Dexibuprofen, Naproxen, Fenoprofen, Ketoprofen, Dexketoprofen, Flurbiprofen, Oxaprozin, Loxoprofen), Acetic acid derivatives, (Indomethacin, Tolmetin, Sulindac, Etodolac, Ketorolac, Diclofenac, Nabumetone), Enolic acid (Oxicam) derivatives (Piroxicam, Meloxicam, Tenoxicam, Droxicam, Lornoxicam, Isoxicam), Fenamic acid derivatives or “Fenamates” (Mefenamic acid, Meclofenamic acid, Flufenamic acid, Tolfenamic acid), Selective COX-2 inhibitors (Celecoxib, Rofecoxib, Valdecoxib, Parecoxib, Lumiracoxib, Etoricoxib, Firocoxib), Sulphonanilides such as Nimesulide, and a range of other compounds (Licofelone, Lysine clonixinate, Hyperforin, Figwort).

Opioids, also known as narcotics, are increasingly recognized as important treatment options for chronic pain. Opioids, along with anticonvulsants and antidepressants are the most consistently effective class of drugs for neuropathic pain. Opioids must be used only in appropriate individuals and under close medical supervision. Several opioids, particularly methadone, and ketobemidone possess NMDA antagonism in addition to their μ-opioid agonist properties. Methadone does so because it is a racemic mixture; only the 1-isomer is a potent μ-opioid agonist. The d-isomer does not have opioid agonist action and acts as an NMDA antagonist; d-methadone is analgesic in experimental models of chronic pain. Clinical studies are in progress to test the efficacy of d-methadone in neuropathic pain syndromes.

### II. A3 ADENOSINE RECEPTORS AND PAIN

The A₃ adenosine receptor (A₃AR) belongs to the Gi-protein-associated cell membrane receptors. Activation of these receptors inhibits adenylate cyclase activity, inhibiting cAMP formation, leading to the inhibition of PKA expression and initiation of a number of downstream signaling pathways. A variety of agonists to this receptor subtype have been synthesized, with IB-MECA (N⁶-(3-iodobenzyl)-adenosine-5′-N-methyluronamide) and its chlorinated form CI-IB-MECA (2-chloro-N⁶-(3-iodobenzyl)-adenosine-5′-N-methyluronamide), believed to be among the most potent and specific presently known A₃AR agonists. Such compounds have shown efficacy in several animal models of inflammation, ischemia, reperfusion injuries, and cancer and have advanced to clinical trial studies for rheumatoid arthritis and cancer.

The present inventor has previously described the use of A₃AR agonists as pharmaceutical compounds in treatments against pain (U.S. Patent Publication 2012/0270829). In particular, A₃AR agonists have been found to be effective in the treatment of neuropathic pain, especially with regard to blocking and/or reversing the development of chemotherapy-induced neuropathic pain (CIPN) and nerve-injury-derived neuropathic pain. Thus, A₃AR agonists were proposed for use in shielding cancer patients from the pain due to chemotherapeutic agents and other causes. Moreover, A₃AR agonists and analgesics have been found to exhibit a synergistic effect in the treatment of neuropathic pain. However, A3AR agonists have no effect on normal pain behavior (i.e., unlike opioids which block acute nociception in response to severe noxious stimuli, for example using a tail flick assay, A3AR agonists have no effect). In addition when given acutely together, an A3AR agonist will not potentiate the antinociceptive effect of an opioid in models of acute nociception.

### III. A₃AR AGONISTS

It can be confirmed that a compound has an A₃AR activity by known methods. Examples of A₃AR agonists that may be used in accordance with the present include, but are not limited to, N⁶-benzyladenosine-5′-N-methyluronamides such as N⁶-(3-iodobenzyl)-adenosine-5′-N-methyluronamide, also known as IB-MECA, and 2-Chloro-N⁶-(3-iodobenzyl)-adenosine-5′-N-methyluronamide (also known as 2-CI-IB-MECA; (N)-methanocarbanucleosides such as (1R,2R,3S,4R)-4-(2-chloro-6-((3-chlorobenzyl)amino)-9H-purin-9-yl)-2,3-di-hydroxy-N-methylbicyclo[3.1.0]hexane-1-carboxamide (also known as CF502, Can-Fite Biopharma, MA); (2S,3S,4R,5R)-3-amino-5-[6-(2,5-dichlorobenzylamino)purin-9-yl]-4-hydroxy-tetrahydrofuran-2-carboxylic acid methylamide (also known as CP-532,903); (1′S,2′R,3′S,4′R,5′S)-4-(2-chloro-6-(3-chlorobenzylamino)-9H-purin-9-yl)-2,3-dihydroxy-N-methylbicyclo[3.1.0]hexane-1-carboxamide (also known as MRS-3558), 2-(1-Hexynyl)-N-methyladenosine; (1S,2R,3S,4R)-2,3-dihydroxy-4-(6-((3-iodobenzyl)amino)-4H-purin-9(5H)-yl)-N-methylcyclopentanecarboxamide (also known as CF101, Can-Fite), (1S,2R,3S,4R)-4-(2-chloro-6-((3-iodobenzyl)amino)-4H-purin-9(5H)-yl)-2,3-dihydroxy-N-methylcyclopentanecarboxamide (also known as CF102, Can-Fite); (1′R,2′R,3′S,4′R,5′S)-4-{2-chloro-6-[(3-iodophenylmethyl)amino]purin-9-yl-}-1-(methylaminocarbonyl)-bicyclo[3.1.0]hexane-2,3-diol (also known as MRS1898); and 2-Dialkynyl derivatives of (N)-methanocarbanucleosides, 2-(arylethynyl)adenine and N(6)-methyl or N(6)-(3-substituted-benzyl), N(6)-methyl 2-(halophenylethynyl) analogues, polyaromatic 2-ethynyl N(6)-3-chlorobenzyl analogues, such as 2-p-biphenylethynyl MRS5679 and fluorescent 1-pyrene adduct MRS5704, as well as MRS5678. Preferred compounds include, but are not limited to, IB-MECA, CF101, and CF102.

Also included are A₃AR allosteric modulators which enhance the receptor activity in the presence of the native ligand, such as 2-cyclohexyl-N-(3,4-dichlorophenyl)-1H-imidazo[4,5-c]quinolin-4-amine (also known as CF602, Can-Fite). However, the above-listed A₃AR agonists are by no means exclusive and other such agonists may also be used. The administration of A.sub.3AR agonists covalently bound to polymers is also contemplated. For example, A₃AR agonists may be administered in the form of conjugates where an agonist is bound to a polyamidoamine (PAMAM) dendrimer. The following table illustrates additional A₃AR agonists that can be employed in accordance with the present invention:

```
  5-7, 17, 18
  8-16
```

| | | | | affinity (K_{V} nM) or % inhibition (in italics)^{a,b} | | | |
|---|---|---|---|---|---|---|---|
| compd | R¹ | R² | species | A₁ | A_{2A} | A₃ | % efficacy,^{c} A₃ |
| 5^{d} | 3-Cl—Bn | H | h | (20% ± 3%) | (27% ± 3%) | 1.34 ± 0.30 | 101 ± 5.9 |
| | | | m | (50% ± 5%) | (2% ± 1%) | 1.23 ± 0.14 | ND |
| 6 | 3-Cl—Bn | 4-SO₃H | h | 383 ± 75 | (23% ± 3%) | 11.1 ± 1.6 | 96.8 ± 5.7 |
| | | | m | 35.1 ± 5.5 | (14% ± 4%) | 9.68 ± 0.15 | 95.7 ± 19.1 |
| 7 | 3-Cl—Bn | 3-SO₃H | h | (16% ± 3%) | (7% ± 6%) | 1.90 ± 0.03 | 98.2 ± 6.7 |
| | | | m | (15% ± 2%) | (1% ± 1%) | 11.3 ± 1.9 | 89.3 ± 7.1 |
| 8^{d} | Me | H | h | (18% ± 1%) | (18% ± 3%) | 5.48 ± 1.23 | 12.6 ± 4.0 |
| | | | m | 3800 ± 780 | (8% ± 3%) | 1530 ± 240 | ND |
| 9^{d} | Et | H | h | (36% ± 4%) | (42% ± 4%) | 5.02 ± 2.19 | 0.8 ± 5.2 |
| | | | m | (49% ± 6%) | (49% ± 2%) | 1480 ± 170 | ND |
| 10^{d} | Et | 2-Cl | h | (25% ± 11%) | (17% ± 6%) | 5.80 ± 2.08 | 7.0 ± 5.2 |
| | | | m | (47% ± 8%) | (11% ± 1%) | (50% ± 9%) | ND |
| 11^{d} | 3-Cl—Bn | H | h | (37% ± 4%)^{g} | 680 ± 170 | 39.0 ± 20.0 | 13.8 ± 5.1 |
| 12^{d} | 3-Cl—Bn | 3-Cl | h | (26% ± 3%) | 1800 ± 310 | 210 ± 40 | 4.5 ± 4.9 |
| 13^{d} | 3-Cl—Bn | 4-Ph | h | (48% ± 4%) | (12% ± 7%) | 54.0 ± 7.0 | 3.5 ± 3.2 |
| | | | m | 1110 ± 220 | (0%) | 255 ± 77 | ND |
| 14^{d} | Ph(CH₂)₂ | H | h | (30% ± 8%) | (22% ± 5%) | 20.0 ± 6.0 | 4.1 ± 1.2 |
| | | | m | (39% ± 6%) | (13% ± 2%) | 480 ± 90 | 14.3 ± 6.1 |
| 15^{d} | Ph₂CHCH₂ | 2-Cl | h | (26% ± 4%) | (22% ± 2%) | 140 ± 30 | 2.6 ± 1.3 |
| | | | m | (23% ± 1%) | (16% ± %) | (54% ± 3%) | ND |
| 16 | 4-SO₃H—Ph(CH₂)₂ | H | h | (10% ± 5%) | (15% ± 3%) | 30.2 ± 4.3 | 7.2 |
| | | | m | (18% ± 2%) | (1% ± 2%) | 3920 ± 1190 | ND |
| 17 | Ph(CH₂)₂ | H | h | (11% ± 3%) | (32% ± 4%) | 1.23 ± 0.57 | 105.3 ± 9.8 |
| | | | m | (25% ± 5%) | (1% ± 1%) | 8.75 ± 2.12 | 114.6 ± 14.6 |
| 18 | 4-SO₃H—Ph(CH₂)₂ | H | h | (9% ± 5%) | (1% ± 1%) | 12.1 ± 1.0 | 93.8 ± 7.1 |
| | | | m | (7% ± 2%) | (0%) | 71.1 ± 13.0 | ND |

^{a}Binding in membranes prepared from CHO or HEK293 (A_{2A} only) cells stably expressing one of three hAR subtypes. The binding affinity for A₁AR, A_{2A}AR, and A₃AR was expressed as K₂ values (n − 3-4) using agonist radioligands [³H]N⁶-R-phenylisopropyladenosine 40, [³H]2-[p-(2-carboxyethyl)phenylethylamino]-5′-N-ethylcarboxamidoadenosine 41, or [^{12S}I]N⁶-(4-amino-3-iodobenzyl)adenosine-5′-N-methyluronamide 42, resepctively. A percent in parentheses refers to inhibition of binding at 10 μM.
^{b}Binding in membranes prepared from HEK293 cells stably expressing one of three mAR subtypes. Radioligand used were [^{12S}I]N⁶-(4-amino-3-iodobenzyl)adenosine-5′-N-methyluronamide 43 (A₁AR and A₃AR) and [³H]2-[p-(2-carboxyethyl)phenylethylamino]-5′-N-ethylcarboxamidoadenosine 41 (A_{2A}AR). The data (n − 3-4) are expressed as K₂ values. A percent in parentheses refers to inhibition of binding at 10 μM.
^{c}Efficacy, expressed as a percentage of the maximal effect of either 5′-N-ethylcarboxamidoadenosine 43 (hA₃ARs) or N⁶-(3-iodobenzyl)adenosine-5′-N-methyluronamide 1a (mA₃ARs) to inhibit forskolin-stimulated cAMP production, was determined in cAMP assays using hA₃AR-transfected CHO cells or mA₃AR-transfected HEK cells. In studies with the hA₃AR, maximal efficacies of 43 and the test compounds were estimated by measuring the extent of inhibition of forskolin-stimulated cAMP accumulation produced each at a concentration of 10 μM. In studies with the mA₃AR, maximal efficacies of 1a and test compounds were determined from concentration-effect curves. Data are expressed as mean ± SEM (n − 3-5).
ND: not determined.
^{d}Compounds 5 and 8-15 were prepared and tested for binding at the hARs in ref 26.

### IV. OPIOIDS FOR USE IN COMBINATION WITH A₃AR AGONISTS

The following is a non-limiting list of opioids that can be administered in combination with A₃AR agonists in accordance with the present invention: Morphine, Opium, Hydromorphone, Nicomorphine, Oxycodone, Dihydrocodeine, Diamorphine, Papaveretum, Codeine, Phenylpiperidine derivatives, Ketobemidone, Pethidine, Fentanyl, Pethidine, Diphenylpropylamine derivatives, Piritramide, Dextropropoxyphene, Bezitramide, Methadone, Dextropropoxyphene, Benzomorphan derivatives, Pentazocine, Phenazocine, Oripavine derivatives, Buprenorphine, Etorphine, Oripavine derivatives, Morphinan derivatives, Butorphanol, Nalbuphine, Tilidine, Tramadol and Dezocine.

### V. PHARMACEUTICAL FORMULATIONS AND ROUTES OF ADMINISTRATION

Where clinical applications in treating pain are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render materials stable and allow for uptake by target cells. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. Such routes include oral, nasal, buccal, rectal, vaginal or topical route. Alternatively, administration may be by orthotopic, transdermal, intradermal, subcutaneous, intramuscular, intraperitoneal, intrathecal or intravenous injection. Such compositions would normally be administered as pharmaceutically acceptable compositions, described supra. Of particular interest is transdermal, intraperitoneal, intravenous or oral administration.

With regard to transdermal delivery, a patch is particularly contemplated. There are five main types of transdermal patches. In the Single-layer Drug-in-Adhesive, the adhesive layer of this system also contains the drug. In this type of patch the adhesive layer not only serves to adhere the various layers together, along with the entire system to the skin, but is also responsible for the releasing of the drug. The adhesive layer is surrounded by a temporary liner and a backing. In Multi-layer Drug-in-Adhesive, the multi-layer drug-in adhesive patch is similar to the single-layer system in that both adhesive layers are also responsible for the releasing of the drug. One of the layers is for immediate release of the drug and other layer is for control release of drug from the reservoir. The multi-layer system is different however that it adds another layer of drug-in-adhesive, usually separated by a membrane (but not in all cases). This patch also has a temporary liner-layer and a permanent backing.

Unlike the Single-layer and Multi-layer Drug-in-adhesive systems, the reservoir transdermal system has a separate drug layer. The drug layer is a liquid compartment containing a drug solution or suspension separated by the adhesive layer. This patch is also backed by the backing layer. In this type of system the rate of release is zero order.

The Matrix system has a drug layer of a semisolid matrix containing a drug solution or suspension. The adhesive layer in this patch surrounds the drug layer partially overlaying it. Also known as a monolithic device.

In Vapor Patches, the adhesive layer not only serves to adhere the various layers together but also to release vapour. The vapour patches are new on the market and they release essential oils for up to 6 hours. The vapour patches release essential oils and are used in cases of decongestion mainly. Other vapour patches on the market are controller vapour patches that improve the quality of sleep. Vapour patches that reduce the quantity of cigarettes that one smokes in a month are also available on the market.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. Subjects

The methods of the invention can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice.

### VI. THERAPIES

A. Treating Pain/Avoiding Tolerance

Treating pain and avoiding tolerance to pain killers are major issues in clinical medicine. One goal of current research is to find ways to improve the efficacy of pain relief, as well as prevent the development of tolerance or addiction, and reduce side effects. One way is by combining such traditional therapies with the therapies of the present invention. In the context of the present invention, it is contemplated that an A₃AR agonist may be used in a combination therapy with an opiate for chronic use.

The therapies would be provided in a combined amount effective to reduce tolerance and to reduce side effects associated with the opioid, including but not limited to addiction and withdrawal. This process may involve contacting the patient with the agents/therapies at the same time. This may be achieved by contacting the patient with a single composition or pharmacological formulation that includes both agents, or by contacting the cell with two distinct compositions or formulations, at the same time, wherein one composition includes the A₃AR agonist and the other includes the opiatte.

Alternatively, the treatment according to the present invention may precede or follow the other treatment by intervals ranging from minutes to weeks. In embodiments where the opiate and the A₃AR agonist are applied separately to the subject, one would generally ensure that a significant period of time did not expire between each delivery, such that the therapies would still be able to exert an advantageously combined effect on the subject. In such instances, it is contemplated that one would administer both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the A₃AR agonist or the opioid therapy will be desired. Various combinations may be employed, where the A₃AR agonist is “A,” and the opioid therapy is “B,” as exemplified below:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations, including chronic and continuous dosing of one or both agents, are contemplated.

B. Treating Symptoms of Withdrawal

Opiate withdrawal refers to the wide range of symptoms that occur after stopping or dramatically reducing opiate drugs after heavy and prolonged use (several weeks or more). Typical opiate drugs for which withdrawal is observed include heroin, morphine, codeine, Oxycontin, Dilaudid, and methadone. Indeed, about 9% of the population is believed to misuse opiates over the course of their lifetime, including illegal drugs like heroin and prescription pain medications such as Oxycontin. These drugs can cause physical dependence, and a person comes to rely on the drug to prevent symptoms of withdrawal. In addition, over time, increasing amounts of the drug become necessary to prevent withdrawal and to provide the needed pain relief or “high” desired by addicts. The amount of time to become physically dependent, and the time need to undergo and outlast withdrawal symptoms varies from person to person.

Typical early symptoms of withdrawal include agitation, anxiety, muscle ache, increased tearing, insomnia, runny nose, sweating, and yawning, while late symptoms of withdrawal include abdominal cramping, diarrhea, dilated pupils, goose bumps, nausea and vomiting. Opioid withdrawal reactions are very uncomfortable but generally are not life threatening. Treatment involves supportive care and medications, such as clonidine, which primarily reduces anxiety, agitation, muscle aches, sweating, runny nose, and cramping. Other medications can treat vomiting and diarrhea.

Drug treatment programs are often used to wean addicts off of their opiate. Methadone, is a synthetic opioid. It is used medically as an analgesic and a maintenance anti-addictive and reductive preparation for use by patients with opioid dependency. Because it is an acyclic analog of morphine or heroin, methadone acts on the same opioid receptors as these drugs, and thus has many of the same effects. Methadone is also used in managing severe chronic pain, owing to its long duration of action, extremely powerful effects, and very low cost. It has cross-tolerance with other opioids including heroin and morphine, offering very similar effects and a longer duration of effect. Oral doses of methadone can stabilise patients by mitigating opioid withdrawal syndrome. Higher doses of methadone can block the euphoric effects of heroin, morphine, and similar drugs. As a result, properly dosed methadone patients can reduce or stop altogether their use of these substances.

Buprenorphine (Subutex®) also has been shown to work for treating withdrawal from opiates, and it can shorten the length of detox. It may also be used for long-term maintenance like methadone. People withdrawing from methadone may be placed on long-term maintenance. This involves slowly decreasing the dosage of methadone over time. This helps reduce the intensity of withdrawal symptoms.

### VII. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Experimental Animals. Morphine Antinociceptive Tolerance Studies:

Male Sprague Dawley rats (200-230 g) were purchased from Harlan (USA) and housed 3-4 per cage and maintained in controlled environment (12 h light/dark cycle) with food and water available ad libitum.

Morphine Withdrawal Studies:

Male Balb/c mice (19-26 g) were purchased from University of Adelaide Laboratory Animal Services (Waite Campus, Adelaidem South Australia) and housed 6 per individually ventilated cage (IVC) and maintained in controlled environment (12 h light/dark cycle) with food and water available ad libitum.

All experiments were performed in accordance with the International Association for the Study of Pain and the National Institutes of Health guidelines on laboratory animal welfare and the recommendations by Saint Louis University Institutional Animal Care and Use Committee (rats) and the National Health and Medical Research Council guidelines on laboratory animal welfare and the recommendations by the University of Adelaide Animal Research Ethics Committee. All experiments were conducted with the experimenters blinded to treatment conditions.

Rat Morphine Antinociceptive Tolerance Studies. Osmotic Pump Implantation.

Male Sprague Dawley rats, were lightly anesthetized with isoflurane and were subcutaneously implanted (in the interscapular region) with primed osmotic minipumps (Alzet 2001; Alza, Mountain View Calif.), to deliver saline at 1 Oh or morphine at 75 μg/μl/h over 7 days as described (King et al., 2007; Vera-Portocarrero et al., 2007). The concentrations of morphine sulfate resulted in a daily dose of approximately 8-9 mg/kg (depending on the weight of the rat). Minipumps were filled according to manufacturer's specifications. The use of the osmotic pump ensures a continuous subcutaneously delivery of morphine avoiding intermittent periods of withdrawal. Rats were tested for analgesia at 2 hr following minipump implant to verify that they are analgesic—approximately 100% analgesia was achieved. This helps verify that the pumps are working well, which is typically not a problem. The integrity of the pump delivery system was reexamined at the end of each experiment when the spinal cords are harvested.

Drug Administration.

The test substance IB-MECA (Tocris, Ellisville, Mo., USA) or its vehicle (3% DMSO in saline) were delivered by intraperitoneal injection (i.p.; 0.2 ml injection volume) between 9-LOAM.

Behavioral Tests. Acute Nociception.

The tail flick test, which measures withdrawal latencies of the tail from a noxious radiant heat source, was used to measure thermal nociceptive sensitivity with baseline latencies of 2-3 sec and a cut-off time of 10 sec to prevent tissue injury (D'Amour, 1941). Tolerance to the antinociceptive effect of morphine was performed as previously described by the inventor's group (Muscoli et al., 2010) and indicated by a significant (P<0.05) reduction in tail flick latency after challenge with an acute dose of morphine sulfate (5 mg/kg, given i.p.) at 30 min post injection time point, a time previously demonstrated to produce maximal antinociception at this dose. Data obtained were converted to percentage maximal possible antinociceptive effect (% MPE) as follows: (response latency−baseline latency)/(cut off latency−baseline latency)×100. Test substances or vehicle were given on day 0 through 6 after completion of the behavioral tests.

Mouse Morphine Withdrawal Studies. Morphine Dependence Dosing Regimen:

Chronic morphine dependence was induced by repeated injections for three consecutive days with an escalating dose schedule (Liu et al., 2011). Mice (n=8 per treatment) received morphine twice daily (morning and afternoon) for 2 days (day 1: 7.5 and 15; day 2: 30 and 30 mg/kg). On the testing day (day 3), a final morphine dose (30 mg/kg) was administered. A group of control mice (n=7 per strain) received an equal number of saline injections over 3 days.

Co-Administration Dosing Regimens:

IB-MECA solution was prepared from the 5 mg from supplier and was dissolved in 5 ml EtOH (drug found to be soluble). EtOH was maintained at 0.02% final drug preparation. Vehicle was 0.02% EtOH in normal saline. IB-MECA dose was 0.1 mg/kg and was administered together with the morphine regimen outlined above.

Naloxone-Precipitated Withdrawal.

A single dose of naloxone (10 mg/kg) was administered to all mice 1 h after the final morphine/saline dose. Immediately after the naloxone injection, animals were placed into individual Plexiglas observation cylinders (25 h×11 w cm) (Nalgene, Scoresby, VIC, Australia). Withdrawal jumping response symptoms were recorded and the frequency of jumps for each mouse was summed over 30 min. Other characteristic opioid withdrawal behaviors were also recorded such as front paw shakes and hunched/prayer postures. All testing was conducted blind to group assignment.

CCI Model of Neuropathic Pain.

Chronic constriction injury to the sciatic nerve of the left hind leg in mice was performed under general anesthesia using the well-characterized Bennett model. Briefly, mice (weighing 25-30 g at the time of surgery) were anesthetized with 3% isoflurane/100% O₂ inhalation and maintained on 2% isoflurane/100% O₂ for the duration of surgery. The left thigh was shaved and scrubbed with Nolvasan, and a small incision (1-1.5 cm in length) was made in the middle of the lateral aspect of the left thigh to expose the sciatic nerve. The nerve was loosely ligated around the entire diameter of the nerve at 3 distinct sites (spaced 1 mm apart) using silk sutures (6.0). The surgical site was dosed with a single muscle suture and a skin clip. Pilot studies established that under our experimental conditions peak mechanoallodynia develops by day 5-7 (D5-D7) following CCI. Test substances or their vehicles were given subcutaneously (s.c), intraperitoneally (i.p.), or orally by gavage (0.1 ml) at peak mechanoallodynia (137).

### Example 2

### Results

IB-MECA Blocks the Development of Morphine-Induced Antinociceptive Tolerance.

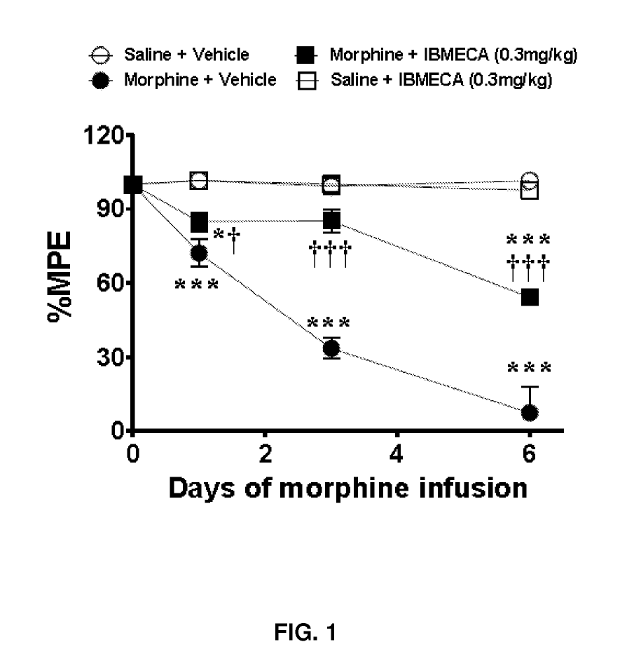

When compared to rats that received a chronic subcutaneous (s.c) infusion of saline (Veh-Sal, n=2) over 7 days, infusion of morphine over the same time frame (Veh-Mor, n=2) led to the development of antinociceptive tolerance indicated by a significant (P<0.001) reduction in tail flick latency 30 min after challenge with an acute dose of morphine (6 mg/kg) given intraperitoneally (i.p) on day 3 and 6 (FIG. 1). Intraperitoneal injection of IB-MECA blocked the development of morphine-induced antinociceptive tolerance (IB-MECA-Sal) (FIG. 1). When given alone daily and over 6 days to rats that received saline infusion (Veh-Sal), IB-MECA (IB-MECA-Sal) had no effect on baseline tail flick latency thus suggesting that activation of the A₃A receptor is not involved in normal pain processing. The ability of IB-MECA to block morphine-induced antinociceptive was not attributable to acute antinociceptive interactions between this drug and acute morphine. Thus i.p administration of IB-MECA (0.3 mg/kg) 15 min before an acute i.p injection of morphine known to produce about 40-50% antinociception at 30 min (3 mg/kg, n=3) did not potentiate the antinociceptive responses to acute morphine (40±2% antinociception to 35+5% antinociception, n=2).

IB-MECA Significantly Reduces Naloxone Precipitated Morphine Withdrawal Behaviors in Mice.

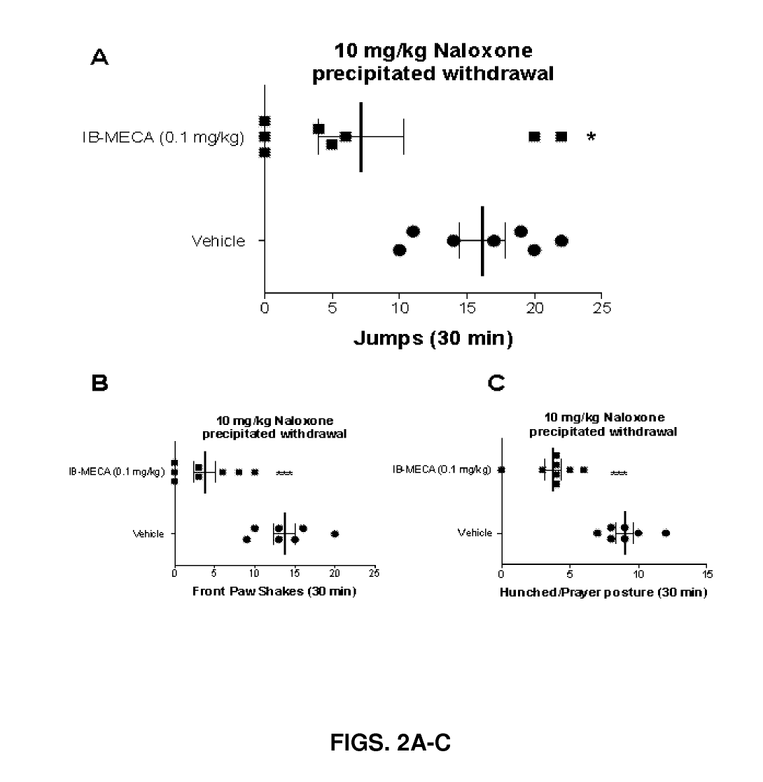

When compared to morphine dependent mice receiving vehicle, IB-MECA treated mice displayed significantly less naloxone-precipitated jumping by 9 jumps/30 min (95% CI 1 to 17), 10 less bouts of front paw shakes/30 min (95% CI 5 to 15) and 5 less episodes of hunched/prayer posture (95% CI 3 to 7) (FIGS. 2A-C).

MRS5698 Prevents Morphine Tolerance.

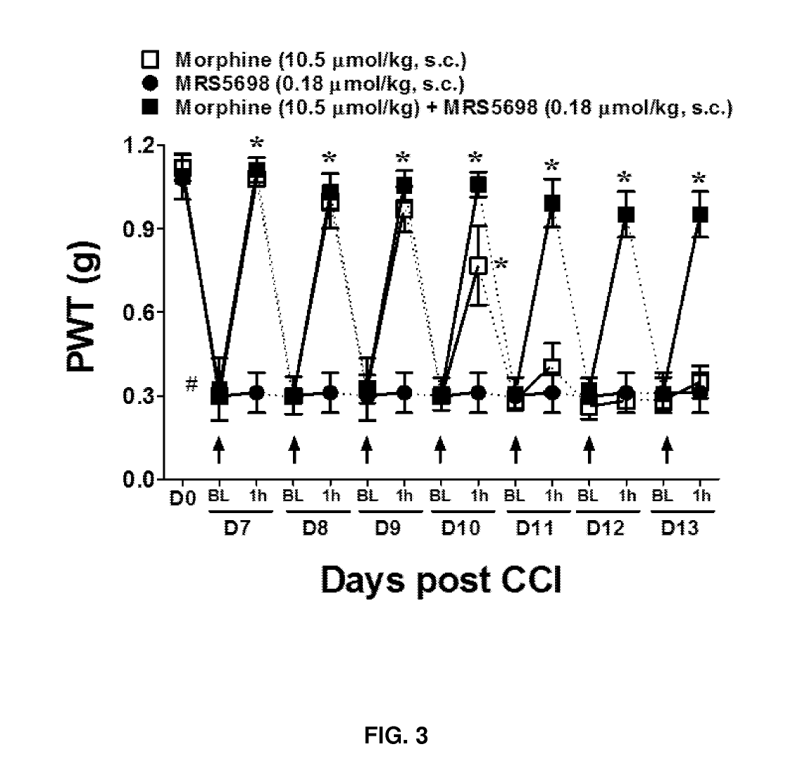

When given at peak pain in rats using a CCI model of neuropathic pain, repeated injections of morphine lose their analgesic effect over time. However, when a non-analgesic dose of MRS5698 is administered along with morphine, the morphine retains is analgesic effects long after control animals lost the analgesic benefit of morphine (FIG. 3).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference:
D'Amour, J Pharmacol xp Ther 72:74-79, 1941.
Foley, Anticancer Drugs 6: Suppl 3, 4-13, 1995.
Fredholm et al., Pharmacological reviews 53:527-552, 2001.
Fredholm et al., Pharmacological reviews 63:1-34, 2011.
King et al., Pain 132:154-168, 2007.
Liu et al., Brain, behavior, and immunity 25:1223-1232, 2011.
Muscoli et al., The Journal of neuroscience: the official journal of the Society for Neuroscience 30:15400-15408, 2010.
Remington's Pharmaceutical Sciences, 15th Edition.
Renfrey et al., Nat Rev Drug Discov 2:175-176, 2003.
Vera-Portocarrero et al., Pain 129:35-45, 2007.

1. A method of reducing opioid antinociceptive tolerance in a subject receiving opiate therapy for acute/severe and chronic neuropathic (malignant and non malignant) pain comprising administering to said subject an amount of an A₃AR agonist sufficient to reduce opioid antinociceptive tolerance.

2. The method of claim 1, wherein said opioid is morphine, oxycodone, or fentanyl.

3. The method of claim 1, wherein said A₃AR agonist is IB-MECA or MRS5698.

4. The method of claim 1, wherein said subject is a human.

5. The method of claim 1, wherein said subject is a non-human mammal.

6. The method of claim 1, wherein said nociceptive pain is chronic pain.

7. The method of claim 1, wherein said nociceptive pain is acute pain.

8. The method of claim 1, wherein said opiate and said A₃AR agonist are delivered at the same time.

9. The method of claim 8, wherein said opiate and said A₃AR agonist are co-formulated.

10. The method of claim 8, wherein said opiate and said A₃AR agonist are not co-formulated.

11-14. (canceled)

15. The method of claim 6, wherein said A₃AR agonist and said opiate are delivered over a period of one week, two weeks, three weeks, four weeks, one month, two months, three months, four months, five months, six months, seven months, eight months, nine months, ten months, eleven months, one year, two years or three years.

16. The method of claim 1, wherein said opiate and/or said A₃AR agonist are delivered by continuous infusion.

17. The method of claim 16, wherein continuous infusion is provided by an implanted pump.

18. The method of claim 1, wherein said nociceptive pain is the result of an injury.

19. The method of claim 18, wherein said injury is a penetration wound, a burn, frostbite or a fracture.

20. The method of claim 1, wherein said nociceptive pain is the result of a disease.

21. The method of claim 20, wherein said disease is diabetes, postsurgical pain, bone cancer pain, spinal nerve injuries, multiple sclerosis, arthritis, an autoimmune disease, or an infection.

22. A method of preventing or treating opioid withdrawal in a subject receiving opiates comprising administering to said subject an amount of an A₃AR agonist sufficient to treat one or more symptoms of opioid withdrawal.

23-40. (canceled)

41. The method of claim 22, wherein said subject is an abuser of illegal or illegally obtained opiate.

42. The method of claim 22, wherein one or more symptoms comprise agitation, anxiety, muscle ache, increased tearing, insomnia, runny nose, sweating, and yawning, while late symptoms of withdrawal include abdominal cramping, diarrhea, dilated pupils, goose bumps, nausea and/or vomiting.

43. The method of claim 22, further comprising subjecting said subject to a drug treatment program.

44. The method of claim 43, wherein said drug treatment program is methadone treatment or buprenorphine treatment.
